(19) United States
(12) Patent Application Publication
(10) Pub. No.: US 2007/0293599 A1
(43) Pub. Date: Dec. 20, 2007

(54) Dental Cement Composition

(75) Inventors: Norikazu Ueyama, Osaka-shi (JP); Hiroshi Mohri, Noda-shi (JP)

Correspondence Address: KENYON & KENYON LLP, ONE BROADWAY, NEW YORK, NY, 10004, US

(73) Assignee: UM DENTAL CO., LTD., TSU-SHI, MIE-KEN (JP)

(21) Appl. No.: 10/555,610

(22) PCT Filed: Apr. 14, 2004
(86) PCT No.: PCT/JP04/05331

§ 371 Date: Nov. 2, 2005

(30) Foreign Application Priority Data
May 7, 2003 (JP) 2003-128982

(51) Int. Cl. A61K 6/087

(52) U.S. Cl. 523/120

(57) ABSTRACT

A dental cement composition comprising an organic polymer and an inorganic powder including a polyvalent metal compound. The polymer comprises a unit (A) containing a (substituted) carboxyl group represented by a formula (I), and a unit (B) containing a (substituted) carbamoyl group represented by a formula (II). A sum of the units (A) and (B) accounts for at least 20 mol % of all units that form the organic polymer and a ratio of the unit (A)/unit (B) in the organic polymer is within a range from 0.6/1.0 to 1.0/0.6. When the quantity of the unit (A) or (B) having a smaller quantity than the other unit within the polymer is deemed 100 mol %, then in at least 70 mol % of the unit (A) or (B), carbons bonded to the (substituted) carboxyl group in the unit (A) and the (substituted) carbamoyl group in the unit (B) are either directly adjacent, or bonded together via a methylene group or ethylene group. 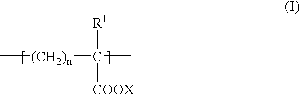
(In formula (I), n represents either 0 or 1, X represents a hydrogen atom, —NH₄, or 1/mM (wherein, M is a metal atom selected from the group consisting of alkali metals, alkali earth metals, transition metals, Zn and Cd, and m represents a valency of the metal), and R¹ represents a hydrogen atom or a methyl group.) 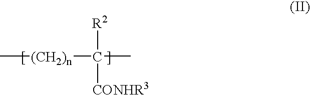
(In formula (II), n represents either 0 or 1, R² represents a hydrogen atom or a methyl group, and R³ represents a hydrogen atom, an alkyl group, alkenyl group, aralkyl group or phosphonoxyalkyl group of 1 to 18 carbon atoms.)

### TECHNICAL FIELD

The present invention relates to a dental cement composition.

Priority is claimed on Japanese Patent Application No. 2003-128982, filed May 7, 2003, the content of which is incorporated herein by reference.

### BACKGROUND ART

Many kinds of dental cements are known, and among them, a glass ionomer cement which uses a reaction between a polycarboxylic acid and a glass such as a fluoroaluminosilicate glass powder has been widely used as the most preferable dental cement recently. The glass ionomer cement is hardened due to an ion reaction between a carboxyl group of the polycarboxylic acid and a metal ion released from the glass, wherein the metal ion is released due to the acid action in the presence of water.

The glass ionomer cement has many characteristics such as excellent affinity, adhesiveness to tooth substrate and durability in an oral cavity, in addition to a characteristic wherein the cement can provide a translucent hardened product having a good appearance. Accordingly, the glass ionomer cement is widely used in many fields such as a coherent for an inlay, crown and the like, a filling and backing for a cavity for caries, a prophylactic filling for a small dental cavity and pit, and the like.

However, the biggest defect of the glass ionomer cement is that, if the glass ionomer cement makes contact with moisture such as saliva and the like in an early stage of the hardening immediately after mixing, the hardening reaction thereof is prevented, and as a result, deterioration of the physical properties of the cement occurs. The hardening reaction of the glass ionomer cement occurs in the presence of water due to a chelate forming reaction between a polycarboxylic acid and a polyvalent metal which is provided from an inorganic compound such as a fluoroaluminosilicate glass. Accordingly, water is used essentially in the reaction for releasing a metal ion. Therefore, on the other hand, water existing in the hardening system or the hardened material has a negative effect on the hardening rate and initial intensity.

Therefore, initial adhesiveness to a tooth substrate of the glass ionomer cement is poor, and other physical properties of the glass ionomer cement also deteriorate with the passage of time.

Moreover, toughness of the glass ionomer cement is particularly inferior to a composite resin and resin cement.

In order to improve the aforementioned defects, developments to obtain a cement which can show stable adhesion have been made by providing a hybrid layer on a surface of dentin as an adhesive resin cement. (For example, Japanese Unexamined Patent Application, First Publication No. 9-255515.) The resin reinforcing type glass ionomer cement comprises a fluoroaluminosilicate glass powder, polycarboxylic acid, water, 2-hydroxyethyl methacrylate (HEMA), a crosslinking agent, and the like. Entanglements of hydrogen bonds are formed between the polycarboxylic acid salt and the HEMA polymer due to a redox catalyst or a photo polymerization catalyst, while the acid-base reaction of the fluoroaluminosilicate glass and the polycarboxylic acid progresses, and then a hardened product is produced with toughness which is characteristic of a polymer substance.

However, the resin reinforcing type glass ionomer cement has a lower ion-reactivity than a conventional glass ionomer cement with respect to the tooth substrate. Accordingly, it is hard to say whether the resin reinforcing type glass ionomer cement can have sufficient adhesive strength. Furthermore, operations for the resin reinforcing type glass ionomer cement are complicated, and therefore performance differences due to the skill differences of each dentist tend to occur.

In view of these circumstances, there are demands for a glass ionomer cement which has considerably better adhesiveness to the tooth substrate and does not require complicated operations.

### DISCLOSURE OF INVENTION

The dental cement composition of the present invention is a dental cement composition which comprises the following organic polymer, and an inorganic powder including a polyvalent metal compound.

The organic polymer comprises a unit (A) containing a (substituted) carboxyl group, which is represented by formula (I) shown below, and a unit (B) containing a (substituted) carbamoyl group, which is represented by formula (II) shown below, wherein a sum of the two units (A) and (B) accounts for at least 20 mol % of all the units that constitute the organic polymer, a ratio of the unit (A)/unit (B) is within a range from 0.6/1.0 to 1.0/0.6, and when the quantity of the unit (A) or (B) having a smaller quantity than the other unit within the polymer is deemed 100 mol %, then in at least 70 mol % of the unit (A) or (B), the carbon bonded. to the (substituted) carboxyl group in the unit (A), and the carbon bonded to the (substituted) carbamoyl group in the unit (B) are either directly adjacent, or bonded together via a methylene group or ethylene group.

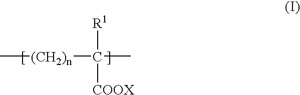

In formula (I), n represents either 0 or 1, X represents a hydrogen atom, —NH₄, or 1/mM (wherein, M is a metal atom selected from the group consisting of alkali metals, alkali earth metals, transition metals, Zn and Cd, and m represents the valency of the metal), and R¹ represents a hydrogen atom or a methyl group.

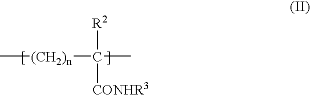

In formula (II), n represents either 0 or 1, R² represents a hydrogen atom or a methyl group, and R3 represents a hydrogen atom, an alkyl group, alkenyl group, aralkyl group or phosphonoxyalkyl group of 1 to 18 carbon atoms.

In the present invention, a (substituted) carboxyl group refers to a carboxyl group itself, and a carboxyl group in which a hydrogen atom has been substituted with —NH₄ or a metal atom selected from the group consisting of the alkali metals, alkali earth metals, transition metals, Zn, and Cd. A (substituted) carbamoyl group refers to a carbamoyl group itself, and a carbamoyl group in which one of the hydrogen atoms has been substituted with an alkyl group, alkenyl group, or aralkyl group of 1 to 18 carbon atoms.

A manufacturing method for a dental cement composition of the present invention comprises, preparing an inorganic powder including a polyvalent metal compound, preparing an organic polymer, and mixing the inorganic powder and the organic polymer to obtain the dental cement composition.

The organic polymer comprises a unit (A) containing a (substituted) carboxyl group, which is represented by formula (I) shown below, and a unit (B) containing a (substituted) carbamoyl group, which is represented by formula (II) shown below, wherein a sum of the two units (A) and (B) accounts for at least 20 mol % of all the units that constitute the organic polymer, the ratio of the unit (A)/unit (B) is within a range from 0.6/1.0 to 1.0/0.6, and when the quantity of the unit (A) or (B) having a smaller quantity than the other unit within the polymer is deemed 100 mol %, then in at least 70 mol % of the unit (A) or (B), the carbon bonded to the (substituted) carboxyl group in the unit (A), and the carbon bonded to the (substituted) carbamoyl group in the unit (B) are either directly adjacent, or bonded together via a methylene group or ethylene group.

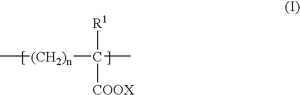

In formula (I), n represents either 0 or 1, X represents a hydrogen atom, —NH₄, or 1/mM (wherein, M is a metal atom selected from the group consisting of the alkali metals, alkali earth metals, transition metals, Zn and Cd, and m represents the valency of the metal), and R¹ represents a hydrogen atom or a methyl group.

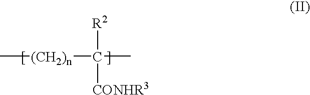

In formula (II), n represents either 0 or 1, R² represents a hydrogen atom or a methyl group, and R³ represents a hydrogen atom, an alkyl group, alkenyl group, aralkyl group or phosphonoxyalkyl group of 1 to 18 carbon atoms.

The aforementioned method of the present invention may further comprise, preparing water, and further mixing the water with the inorganic powder and the organic polymer.

### BEST MODE FOR CARRYING OUT THE INVENTION

A description of preferred examples of the present invention follows. However, the present invention is not limited by any of the following examples, and may include, for example, suitable combinations of structural elements from the various examples.

The present invention relates to a dental cement composition. Objects of the present invention include to improve faults of a conventional glass ionomer cement, and to provide a cement composition which has an excellent initial strength, hardening speed, and adhesiveness to a tooth substrate, and to provide a cement composition in which deterioration of properties thereof does not occur with the passage of time.

A dental cement composition of the present invention uses a new organic polymer as an adhesive component for the tooth substrate, instead of a conventional polycarboxylic acid. The new organic polymer includes a unit wherein a unit (A) containing a (substituted) carboxyl group and a unit (B) containing a (substituted) carbamoyl group are bonded adjacently. Here, the term “(substituted)” used above indicates the presence of a substituent(s) (which is deemed to also include the case of a hydrogen atom as a substituent).

The organic polymer used in a dental cement composition of the present invention is a polymer in which the sum of the two units (A) and (B) is at least 20 mol %, and preferably at least 40 mol %, and even more preferably 60 mol % or more, of all the units that constitute the polymer. Of the various possible polymers, a polymer formed solely from the units (A) and (B), namely, a polymer in which the above sum is 100 mol %, is the most preferred.

When the sum of the units (A) and (B) is less than 100%, then there are no particular restrictions on the other units that may be incorporated within the organic polymer. Any appropriate units may be incorporated. Examples of these other units include alkylene units, cycloalkylene units, and units with amide linkages, each of which may contain an alkyl group, an aromatic group, and/or an alkyloxycarbonyl group as a side chain.

The units (A) and (B) must exist within the organic polymer in a ratio (A):(B) within a range from 0.6:1.0 to 1.0:0.6.

Provided the sum of the units (A) and (B) accounts for at least 20 mol % of the polymer, and the ratio (A):(B) falls within the required range listed above, a structure (C) as shown in formula (III) below, wherein the carbon bonded to the (substituted) carboxyl group of the unit (A), and the carbon bonded to the (substituted) carbamoyl group of the unit (B) are either directly adjacent, or bonded together via a methylene group or ethylene group, can be formed efficiently.

The units (A) and (B) must exist within the organic polymer in a ratio (A):(B) within a range from 0.6:1.0 to 1.0:0.6. Provided the sum of the units (A) and (B) accounts for at least 20 mol % of the polymer, and the ratio (A) : (B) falls within the required range listed above, the structure (C) such as that shown in formula (III) below, wherein the carbon bonded to the (substituted) carboxyl group of the unit (A), and the carbon bonded to the (substituted) carbamoyl group of the unit (B) are either directly adjacent, or bonded together via a methylene group or ethylene group, can be formed efficiently.

When the structure (C) is formed, bonding of the organic polymer and the inorganic compound can become strong, and therefore, the dental cement composition comprising the polymer which includes the structure (C) can be adhered to the tooth substrate strongly. In order to form the structure (C), it is preferable that the amounts of unit (A) and unit (B) existing in the polymer are approximately the same level.

The ratio (A):(B) is preferably from 0.7:1.0 to 1.0:0.7, and even more preferably from 0.8:1.0 to 1.0:0.8. Polymers in which the respective quantities are equal are also very desirable. 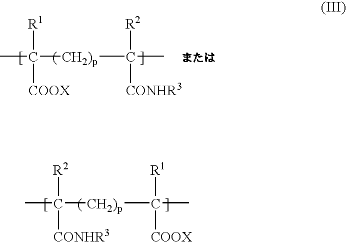
(In formula (III), p represents 0, 1, or 2, X represents a hydrogen atom, —NH₄, or 1/mM (wherein, M is a metal atom selected from a group consisting of the alkali metals, alkali earth metals, transition metals, Zn and Cd, and m represents the valency of the metal), R¹ represents a hydrogen atom or a methyl group, R² represents a hydrogen atom or a methyl group, and R³ represents an alkyl group, alkenyl group, aralkyl group or phosphonoxyalkyl group of 1 to 18 carbon atoms.)

In addition, when the quantity of one of the units (A) and (B) within the polymer is deemed 100 mol % and said one unit exists in smaller amounts than the other within the polymer, then in at least 70 mol %, and preferably in at least 80%, and even more preferably in at least 90%, and most preferably in 100%, of the one unit, the carbon bonded to the carboxyl group in the unit (A) and the carbon bonded to the carbamoyl group in the unit (B) must form a structure (C), wherein the two carbon atoms are either directly adjacent, or bonded together via a methylene group or ethylene group.

For example, in the case in which the unit (A) accounts for 40 mol %, and the unit (B) accounts for 30 mol %, of the 100 mol % of units that constitute the polymer, the quantity of the unit (B), which is the lesser of the two units, is used as the standard. The above requirement means that at least 70 mol % of the unit (B) (namely, at least 21 mol % of all the structural units) must exist adjacent to unit (A), forming a structure (C).

Provided the structure (C) is formed in the above quantities, the adhesion between the dental cement composition containing the polymer and the tooth substrate is increased.

Furthermore, it is preferable that the structure (C) included in the organic polymer of the present invention can be distributed relatively evenly throughout the polymer molecular chain of the organic polymer of the present invention by conducting, for example, a production method described below, and that an irregularly distributed configuration is not formed in which there is one portion wherein the structure (C) is concentrated and another portion wherein the structure (C) does not exist at all.

It is thought that, when the structure (C) is distributed evenly as described above, then the dental cement composition should bond even more strongly to the tooth substrate. If the organic polymer is produced using the method described below, then the structure (C) can be distributed comparatively evenly through out the polymer molecule.

In the structure (C), the oxygen atom of the —C═O group of the (substituted) carboxyl group and the hydrogen atom of the (substituted) carbamoyl group form an intramolecular hydrogen bond. It is thought that the existence of this hydrogen bond strengthens the acid strength of the carboxyl group, enabling powerful bonding of the carboxyl group to the calcium within the tooth substrate. It is thought that the aforementioned intramolecular hydrogen bond is formed with a COO⁻ anion derived from a carboxyl group.

For example, examples of benzoic acids which do not have a carbamoyl group in close proximity to the carboxyl group are shown below. The pKa value for 2,2-dimethyl propanoyl amino benzoic acid is 5.4, and the pKa value for 2,4,6-trimethyl benzoic acid, which exhibits steric hindrance, is 4.8. In contrast, 2-(2,2-dimethyl propanoyl)-6-methyl benzoic acid, which has a carbamoyl group in close proximity to the carboxyl group, has a stronger acid value, that is it has a pKa value of 3.9, and 2,6-di(2,2-dimethyl propanoyl) methyl benzoic acid, which has carbamoyl groups on both sides of the carboxyl group, has an even stronger acid value, with a pKa value of 3.1. It is thought that when a carbamoyl group exists in the vicinity of the carboxyl group, an intramolecular hydrogen bond is formed, and the formation of this hydrogen bond causes an increase in the acid strength of the carboxyl group.

In terms of promoting the hydrogen bonding and increasing the acid strength, R³ is preferably an alkyl group, alkenyl group, or aralkyl group.

In terms of ease of formation of the aforementioned intramolecular hydrogen bond, the value of p in the structure (C) is preferably either p=0 (wherein the carbon bonded to the (substituted) carboxyl group of the unit (A), and the carbon bonded to the (substituted) carbamoyl group of the unit (B) are directly adjacent to one another) or p=1 (wherein the carbon bonded to the (substituted) carboxyl group of the unit (A), and the carbon bonded to the (substituted) carbamoyl group of the unit (B) are bonded together via a methylene group). A single organic polymer molecule may simultaneously include a structure for which p=0, a structure for which p=1, and/or a structure for which p=2.

The polymerization degree of the organic polymer is preferably at least 5, and even more preferably 10 or greater, and even more preferably 50 or more, and most preferably 100 or more.

A higher polymerization degree is favorable. When an adhesive layer adhering to the tooth substrate is formed using an organic polymer having a higher polymerization degree, the obtained adhesive layer becomes a very tough resin layer, and the layer can work, between a living tissue and an artificial material provided thereon, as a protective layer for blocking external stimuli at the interface. Furthermore, from the viewpoints of ease of preparation of the dental cement composition, and production ability, the polymerization degree of the organic polymer is preferably 10,000 or less. Here, the term “polymerization degree” refers to the number average polymerization degree.

For example, the organic polymer used in the dental cement composition of the present invention can be produced by the method described below.

First, a carboxyl group-containing polymer, in which (meth)acrylic acid units account for at least 20 mol % of all the units that constitute the polymer, is produced by normal methods using a carboxyl group-containing monomer such as (meth)acrylic acid.

Subsequently, the thus obtained carboxyl group-containing polymer is treated with a dehydration agent to convert adjacent sets of 2 carboxyl groups into acid anhydride structures and generate a polymer having introduced acid anhydride groups.

These acid anhydride groups are then subjected to a ring-opening addition under conventional conditions, using an amidation agent such as ammonia or an alkylamine, thereby forming monoamide groups. This reaction can generate an organic polymer that contains the structure (C), wherein the carbon bonded to a (substituted) carboxyl group and the carbon bonded to a (substituted) carbamoyl group are either directly adjacent, or bonded together via a methylene group or ethylene group.

The structure (C) takes account of a case wherein head-head (tail-tail) bonding is formed as well as a case wherein head-tail bonding is formed. That is, when the structure (C) is formed after polymerization of a carboxyl group-containing monomer such as (meth)acrylic acid, at the polymerization, head-head (tail-tail) bonding and/or head-tail bonding may be formed. Those cases in which p is either 0 or 2 represent cases of head-head (tail-tail) bonding, whereas the case in which p=1 represents head-tail bonding.

In an alternative method, an organic polymer usable in a dental cement composition of the present invention can be produced by forming a polymer by conventional methods using maleic anhydride, and then conducting a ring-opening addition of acid anhydride groups of the polymer under conventional conditions, using an amidation agent such as ammonia or an amine.

Because this organic polymer is synthesized entirely from a monomer containing an acid anhydride group, such as maleic anhydride, the value of p in the resulting structure (C) is 0; that is, the carbon atom bonded to the (substituted) carboxyl group, and the carbon atom bonded to the (substituted) carbamoyl group must be bonded together directly within the main polymer chain.

Accordingly, when synthesizing the organic polymer, provided a monomer containing an acid anhydride group is used, the structures (C) in which p is 0 can be reliably produced.

In the polymerization of a monomer containing an acid anhydride group, another monomer capable of copolymerization with maleic anhydride can also be used.

In polymers formed using maleic anhydride, the maleic anhydride unit becomes the unit (C) in the polymer. When the provided maleic anhydride accounts for at least 70% of the polymer, the polymer may be a random copolymer. However, if the quantity of maleic anhydride is within a range 40 to 70%, then in order to ensure that the structure (C) is distributed evenly, an alternating copolymer in which the maleic anhydride portions are distributed evenly is preferred.

An alternating copolymer can be obtained by a radical polymerization of a maleic anhydride and an electron-donating monomer.

Electron-donating monomers are monomers wherein the characteristic e-value is negative. Examples thereof include allyl monomers such as allyl alcohol, vinyl ether monomers such as methyl vinyl ether and ethyl vinyl ether, cyclic ether monomers such as p-dioxene, vinyl ester monomers such as vinyl acetate, α-olefins such as propylene, and styrenes.

The dental cement composition of the present invention comprises an inorganic powder which contains a polyvalent metal compound, in addition to the aforementioned organic polymer. Any polyvalent metal compound can be selected as the polyvalent metal compound of the present invention if needed. Examples thereof include oxides, hydroxides, carbonates, sulfates, phosphates, silicates, and the like of a polyvalent metal. Examples of the oxides of the polyvalent metal include zinc oxide, magnesium oxide, calcium oxide, aluminum oxide, and strontium oxide. Examples of the hydroxide of the polyvalent metal include zinc hydroxide, calcium hydroxide, and aluminum hydroxide. Examples of the carbonate of the polyvalent metal include zinc carbonate, calcium carbonate, aluminum carbonate, and strontium carbonate. Examples of the sulfate of the polyvalent metal include gypsum and barium sulfate. Examples of the phosphate of the polyvalent metal include calcium phosphate, zinc phosphate, and aluminum phosphate. Examples of the silicates include aluminum silicate, calcium silicate, and aluminum borosilicate. Among them, an especially preferable material is a polyvalent metal ion extractable glass which belongs to silicates, and examples thereof include an aluminosilicate glass and a fluoro alumino silicate glass. However, the material is not limited thereto. These compounds can be used independently or in combination of two or more.

When the inorganic powder containing the polyvalent metal compound is used, a strong bond can be provided, because a metal such as aluminum, calcium and the like contained in the powder coordinates with a carboxyl group in the organic powder, the carboxyl group having a strong acid strength strengthened by the intramolecular hydrogen bond.

A mass average particle diameter of the inorganic powder containing the polyvalent metal compound is preferably 10 μm or less.

The inorganic powder may contain a compound other than the polyvalent metal compound. For example, the inorganic powder may contain oxide, hydroxide, carbonate, sulfate, phosphate, and silicate of an alkaline metal, oxide, hydroxide, carbonate, sulfate, phosphate, silicate of a transition metal and the like.

When an adhesion component in the cement composition consists of only the aforementioned organic polymer, the organic polymer can be used such that the organic polymer is dissolved in water or in a mixed solvent of water and an organic solvent to prepare a solution.

The dental cement composition of the present invention may further comprise a (meth)acrylate-based polymerizable monomer and a polymerization catalyst.

When the (meth)acrylate-based polymerizable monomer is contained in the cement composition, for example, an organic polymer, a polymerization catalyst, and the like may be dissolved in the (meth)acrylate-based polymerizable monomer to prepare a solution. Examples of the organic solvent which can be used for forming a solution include ethyl alcohol, propyl alcohol, acetone, and the like, but they are not limited thereto.

Examples of the (meth)acrylate-based polymerizable monomer include: (meth)acrylic acid, hydroxymethyl (meth)acrylate, 2-hydroxyethyl (meth)acrylate, hydroxypropyl (meth)acrylate, hydroxybutyl (meth)acrylate, and a hydroxyhexyl (meth)acrylate; mono- or di-glycerol ester of (meth)acrylic acid; mono-, di-, tri-pentaerythritol ester of (meth)acrylic acid; (meth)acryloyloxyethyl phosphate, (meth)acryloyloxypropyl phosphate, (meth)acryloyloxybutyl phosphate, tetra (meth)acryloyloxyethyl pyrophosphate, methyl (meth)acrylate, n-propyl (meth)acrylate, isopropyl (meth)acrylate, phenyl (meth)acrylate, benzyl (meth)acrylate, 2-ethyl hexyl (meth)acrylate, n-butyl (meth)acrylate, isobutyl (meth)acrylate, decyl (meth)acrylate, lauryl (meth)acrylate, stearyl (meth)acrylate, glycidyl (meth)acrylate, glyceryl (meth)acrylate, diethyl amino ethyl (meth)acrylate, 2,2-bis[(meth)acryloxy polyethoxy phenyl] propane, and 2,2′-bis [4-(3-(meth) acryloyloxy-2-hydroxy propoxy) phenyl] propane. These compounds can be used singly or in combination of two or more.

A photopolymerization catalyst, a redox-polymerization catalyst, and the like can be used as the polymerization catalyst. These catalysts can also be used in combination. Another catalyst may also be used if needed.

Examples of the photopolymerization catalyst include camphoroquinone, naphthoquinone, benzyl, and biacetyl.

Examples of the redox-polymerization catalyst include a combination type catalyst of a known reducing agent and a radical polymerization initiator, such as benzoyl peroxide, cumene hydroperoxide, t-butyl hydroperoxide, potassium persulfate, ammonium persulfate, azobisisobutyronitrile, and azobisvaleronitrile. Examples of the reducing agent include amino-type reducing agents such as N,N-dimethylamino-p-toluidine, butyl diethanolamine, N,N-dimethyl amino ethyl methacrylate, morpholinoethyl methacrylate, and dimethyl aminobenzoate.

These catalysts can be used singly or in combination of two or more.

The cement composition of the present invention can comprise the aforementioned organic polymer, inorganic powder containing a polyvalent metal compound, and water. Polyvalent metal ion is eluted to the surface in the presence of water from the inorganic powder which contains the polyvalent metal compound. The organic polymer can bond with the polyvalent metal ion strongly, since the acid strength of the carboxyl group of the organic polymer is strong. Then, via the bonding, the organic polymer bonds to the inorganic powder strongly, and the organic polymer also bonds to the calcium of the tooth substrate. Accordingly, the dental cement composition of the present invention can bond to the tooth substrate strongly without requiring pretreatment of the surface of the tooth substrate for adhesion, and therefore many steps in operations are not required at the time of applying a filling and/or coherent.

Furthermore, the manufacturing method for the cement composition of the present invention can be suitably selected as required. One example of a preferable manufacturing method involves first mixing water with the organic polymer, and then mixing the mixture uniformly with the inorganic powder containing a polyvalent metal compound at the time of use.

The ratio and the like of the amounts of the inorganic powder containing the polyvalent metal compound and water can be determined arbitrarily. The preferable range of the ratio of the inorganic powder to water is 1:2 to 20:1 (weight ratio).

A preferable concentration of the organic polymer in the mixture of the organic polymer and water is about 10 to 90% by weight.

Furthermore, when a polymerizable monomer and polymerization catalyst are contained therein, the polymerizable monomer can be polymerized under the condition that the monomer and the organic polymer are intertwined. Therefore, it is preferable that the composition is applied to the tooth substrate, since toughness is further provided to the hardened product of the dental organic compound of the present invention.

The dental cement composition of the present invention can be used suitably for a coherent between a teeth structure and a dental prosthetic such as an inlay, a crown, and a bridge, a coherent between a teeth structure and an orthodontic appliance, filling of a caries cavity or groove, relining, and prophylactic filling a small cavity and a pit of a tooth and the like.

### EXAMPLES

A more detailed description of the present invention follows using a series of examples, although the present invention is in no way limited to these examples.

Here, evaluations of mixtures of the dental cement composition obtained by examples and comparative examples were conducted according to the following procedures.

(The Adhesive Strength to Dentin of Bovine Teeth)

A bovine tooth was embedded in wax, and was polished with #600 waterproof emery paper to expose the dentin surface of the bovine teeth. A hole having a diameter of 4 mm was made on an adhesive tape, which was stuck on the surface of the dentin to determine the adhesion area. A mixed dental cement composition was applied to the hole portion, and then, the hole portion was pressed with a round bar which is a hardened cement material having a diameter of 6 mm and a length of 12 mm. The round bar had a hook which was used for a following adhesion test and embedded in advance before the cement had hardened. Then, the tooth was exposed to light for 20 seconds with a light exposure machine. In this way, 20 test pieces were prepared. These test pieces were maintained in an atmosphere of 37° C. and RH of 100% for one hour, and then, immersed in water of 37° C. for 48 hours. Then, ten test pieces were subjected to a tensile test to determine the adhesive strength at a crosshead speed of 1 mm/minute using an Autograph AGS-500 (manufactured by Shimadzu Corporation). Another ten test pieces were subjected to a thermal recycling test wherein the test pieces were immersed 1000 times in cold water of 5° C. and hot water of 55° C. alternatively each for 30 seconds.

(Compressive Strength)

A predetermined mold made of polytetrafluoroethylene was filled with a mixed dental cement composition, and maintained for 30 minutes at room temperature to harden the composition. Then, test pieces used for the compressive test having a diameter of 6 mm and a length of 12 mm were prepared. After immersing the test pieces in water of 37° C. for 48 hours, the compressive test was conducted at a crosshead speed of 2 mm/minute using an Autograph.

### Example 1

Polyacrylic acid (21.6 g) (Mw: 450,000) was dissolved in 1 l of methanol. Furthermore, 55.6 g of tri-n-butylamine was added thereto at room temperature under constant stirring, and then the mixture was stirred for a further 12 hours. Subsequently, the methanol was removed at 50° C. under reduced pressure conditions, using an evaporator, and the residue was then dissolved in 2 l of dichloromethane. Phosphorus nitride chloride cyclic trimer (52.1 g) was added to this solution at 30° C. under constant stirring, and the mixture was then stirred for 12 hours. Subsequently, 2 l of diethyl ether was added to the solution, thereby precipitating a solid.

The solid was isolated by filtration, washed with a small quantity of dichloromethane, and then dried, yielding 16 g of a polyacrylic acid anhydride. As shown below, the generation of an acid anhydride group was confirmed by IR analysis.
IR (KBr): 1804 cm⁻¹ (C═O), 1760 cm⁻(C═O), 1030 cm⁻¹ (CO—O—CO)

The polyacrylic anhydride (6.4 g) was added to 87.8 g of t-butylamine at room temperature, and the resulting mixture was stirred for 12 hours. Following evaporation to a dry solid using an evaporator, the solid was dissolved in 100 g of water. Concentrated hydrochloric acid was then added dropwise to the solution to adjust the pH to 2 to 3. The precipitated solid was collected by filtration, and washed with diethyl ether. The results of subsequent elemental analysis and ¹H-NMR analysis are shown below.
Elemental analysis: (C₁₀H₁₇NO₃)
Calculated values: C 60.28 mol %, N 7.03 mol %, H 8.60 mol %
Measured values: C 60.52 mol %, N 7.12 mol %, H 7.82 mol %
¹H-NMR (DMSO-d₆): 12.00 ppm (COOH), 6.9 ppm (CONH), 1.17 ppm (CH₃)

From these results, it was determined that an organic polymer A had been obtained in which an acrylic acid unit and N-t-butylacrylamide unit were arranged alternately.

Integration of the ¹H-NMR chart revealed a ratio between —COOH and —CONH within the organic polymer A of 1.00:0.88. Furthermore, under these reaction conditions, it was believed that 100 mol % of the amide-based units of the polymer A had formed a structure (C).

Ethyl dimethyl amino benzoate (0.1 g) was mixed with 50 g of a fluoroaluminosilicate glass powder to prepare a cement powder.

On the other hand, 5 g of the organic polymer A obtained as described above was dissolved in a mixed liquid of 1 g of water and 20 g of 2-hydroxy ethyl methacrylate to prepare a solution, and 0.1 g of camphoroquinone was further dissolved in the solution uniformly to prepare a cement liquid.

The cement liquid (1.0 g) was added to 1.8 g of the aforementioned cement powder, and mixing thereof was conducted for 30 seconds to prepare a mixed dental cement composition.

Using the mixed dental cement composition, the adhesive strength of the cement composition to dentin of bovine teeth and the compressive strength of the cement composition were evaluated. The results are shown in Table 1.

### Example 2

Forty parts by mass aqueous solution of the organic polymer A, which was prepared similarly to Example 1, was prepared as a cement liquid.

The cement liquid (1.0 g) was mixed with 1.8 g of the cement powder, which was prepared similarly to Example 1, and mixing was conducted for 30 minutes to prepare a mixed dental cement composition.

The adhesive strength to dentin of bovine teeth and the compressive strength of the cement composition were evaluated using the mixed dental cement composition. The results are shown in Table 1.

### Comparative Example 1

Ammonium persulfate (0.36 g) and 50 mg of hydroquinone were added to 8 g of a mixture of monomers (acrylic acid/acryloyloxyethyl phosphate=95/5 (mass ratio)), and the mixture was put into a glass tube. After the glass tube was sealed subsequent to the replacement of the atmosphere in the tube with nitrogen, polymerization was conducted at 80° C. for 15 hours. The obtained copolymer was subjected to dialysis, freeze-drying, and grinding to obtain a copolymer powder.

Ammonium persulfate (0.1 g) was dissolved uniformly in a solution wherein 5 g of the copolymer powder obtained was dissolved in 20 g of 2-hydroxy ethyl methacrylate to prepare a cement liquid.

The cement liquid (1.0 g) obtained was mixed with 1.8 g of cement powder which was obtained similarly to Example 1 to obtain a mixed dental cement composition.

The adhesive strength to dentin of bovine teeth and the compressive strength were evaluated using the mixed dental cement composition. The results are shown in Table 1.

### Comparative Example 2

Forty parts by mass aqueous solution wherein a copolymer powder which was prepared similarly to comparative example 1 was dissolved in water was prepared. The aqueous solution (1.0 g) as a cement liquid was mixed for 30 seconds with 1.8 g of a cement powder obtained similarly to Example 1 to obtain a mixed dental cement composition.

The adhesive strength to dentin of bovine teeth and the compressive strength were evaluated using the mixed dental cement composition. The results (averages) are shown in Table 1.

TABLE 1

| | Adhesive strength to dentin of | | |
|---|---|---|---|
| | bovine teeth (MPa) | | |
| | After being | | |
| | maintained in | | |
| | water for 48 | After conducting | Compressive |
| | hours | hot-cold cycles | strength (MPa) |
| Example 1 | 19.2 | 18.9 | 120.5 |
| Example 2 | 13.8 | 7.41 | 107.4 |
| Comparative | 7.62 | 4.66 | 102.7 |
| Example 1 | | | |
| Comparative | 3.68 | 2.05 | 90.2 |
| Example 2 | | | |

As shown in the Table 1, the dental cement composition of the present invention comprising the specific organic polymer has higher adhesiveness to a tooth substrate compared with the conventional glass ionomer cement wherein an acrylic acid/acryloyloxyethyl phosphate copolymer is used. Furthermore, as is apparent from the results of the compressive strength test, the cement composition of the present invention is excellent in toughness, and also excellent in water resistance and durability.

### INDUSTRIAL APPLICABILITY

The present invention provides a dental cement composition comprising a specific organic polymer, a metal oxide, and a glass powder. By the present invention, an excellent dental cement composition is provided which is excellent in adhesiveness to tooth substrates, hardening rate, and strength in a short time, and which does not show deterioration of properties with the passage of time.

Furthermore, the dental cement composition of the present invention is excellent in adhesiveness to tooth substrates, hardening rate, and strength in short time, and does not show deterioration of properties with the passage of time, compared with a conventional glass ionomer cement using a polyacrylic acid and a copolymer of an acryloyloxy group-containing monomer and an acrylic acid.

1. A dental cement composition comprising an organic polymer and an inorganic powder including a polyvalent metal compound, wherein
the organic polymer comprises a unit (A) containing a (substituted) carboxyl group represented by formula (I) below, and a unit (B) containing a (substituted) carbamoyl group represented by a formula (II) below;
a sum of the units (A) and (B) accounts for at least 20 mol % of all units that constitute the organic polymer;
a ratio of the unit (A)/unit (B) in the polymer is within a range from 0.6/1.0 to 1.0/0.6; and
when the quantity of the unit (A) or (B) having a smaller quantity than the other unit within the polymer is deemed 100 mol %, then in at least 70 mol % of the unit (A) or (B), a carbon bonded to the (substituted) carboxyl group in the unit (A), and a carbon bonded to the (substituted) carbamoyl group in the unit (B) are either directly adjacent, or bonded together via a methylene group or ethylene group 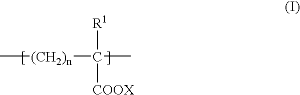
(in formula (I), n represents either 0 or 1, X represents a hydrogen atom, —NH₄, or 1/mM (wherein, M is a metal atom selected from the group consisting of alkali metals, alkali earth metals, transition metals, Zn and Cd, and m represents a valency of the metal), and R¹ represents a hydrogen atom or a methyl group) 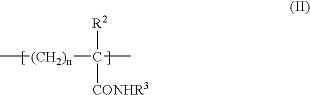
(in formula (II), n represents either 0 or 1, R² represents a hydrogen atom or a methyl group, and R3 represents a hydrogen atom, an alkyl group, alkenyl group, aralkyl group or phosphonoxyalkyl group of 1 to 18 carbon atoms).

2. A dental cement composition according to claim 1, wherein the dental cement composition further comprises water.

3. A dental cement composition according to claim 1, wherein the dental cement composition further comprises a (meth)acrylate-based polymerizable monomer and a polymerization catalyst.

4. A dental cement composition according to claim 1, wherein a sum of the units (A) and (B) accounts for at least 40 mol % of all units that constitute the organic polymer.

5. A dental cement composition according to claim 1, wherein the ratio of the unit (A)/unit (B) is within a range from 0.7/1.0 to 1.0/0.7.

6. A dental cement composition according to claim 1, wherein when the quantity of the unit (A) or (B) having a smaller quantity than the other unit within the polymer is deemed 100 mol %, then in at least 80 mol % of the unit (A) or (B), a carbon bonded to the (substituted) carboxyl group in the unit (A), and a carbon bonded to the (substituted) carbamoyl group in the unit (B) are either directly adjacent, or bonded together via a methylene group or ethylene group.

7. A dental cement composition according to claim 1, wherein R³ is at least one group selected from the group consisting of alkyl groups, alkenyl groups, and aralkyl groups.

8. A dental cement composition according to claim 1, wherein the polyvalent metal compound is at least one selected from the group consisting of oxides, hydroxides, carbonates, sulfates, phosphates, silicates of polyvalent metal.

9. A dental cement composition according to claim 1, wherein the polyvalent metal compound is a polyvalent metal ion extractable glass.

10. A dental cement composition according to claim 3, wherein the (meth)acrylate based polymerizable monomer is at least one selected from the group consisting of (meth)acrylic acid, hydroxy methyl (meth)acrylate, 2-hydroxy ethyl (meth)acrylate, hydroxy propyl (meth)acrylate, hydroxy butyl (meth)acrylate, hydroxy hexyl (meth)acrylate, mono- and di-glycerol ester of (meth)acrylic acid, mono-, di-, and tri-pentaerythritol ester of (meth)acrylic acid, (meth)acryloyloxyethyl phosphorate, (meth)acryloyloxypropyl phosphorate, (meth)acryloyloxybutyl phosphorate, tetra (meth)acryloyloxyethyl pyrophosphate, methyl (meth)acrylate, n-propyl (meth)acrylate, isopropyl (meth)acrylate, phenyl (meth)acrylate, benzyl (meth)acrylate, 2-ethyl hexyl (meth)acrylate, n-butyl (meth)acrylate, isobutyl (meth)acrylate, decyl (meth)acrylate, lauryl (meth)acrylate, stearyl (meth)acrylate, glycidyl (meth)acrylate, glyceryl (meth)acrylate, diethyl amino ethyl (meth)acrylate, 2, 2-bis[(meth)acryloxy polyethoxy phenyl] propane, and 2,2′-bis [4-(3-(meth) acryloyloxy-2-hydroxy propoxy) phenyl] propane.

11. A dental cement composition according to claim 3, wherein the polymerization catalyst is at least one of a redox catalyst and a photo polymerization catalyst.

12. A manufacturing method for a dental cement composition, comprising:
preparing an inorganic powder including a polyvalent metal compound,
preparing an organic polymer, and
mixing the inorganic powder and the organic polymer to obtain the dental cement composition, wherein
the organic polymer comprises a unit (A) containing a (substituted) carboxyl group represented by formula (I) below, and a unit (B) containing a (substituted) carbamoyl group represented by formula (II) below;
a sum of the units (A) and (B) accounts for at least 20 mol % of all units that form the organic polymer;
a ratio of the unit (A)/unit (B) in the organic polymer is within a range from 0.6/1.0 to 1.0/0.6; and
when the quantity of the unit (A) or (B) having a smaller quantity than the other unit within the polymer is deemed 100 mol %, then in at least 70 mol % of the unit (A) or (B), a carbon bonded to the (substituted) carboxyl group in the unit (A), and a carbon bonded to the (substituted) carbamoyl group in the unit (B) are either directly adjacent, or bonded together via a methylene group or ethylene group 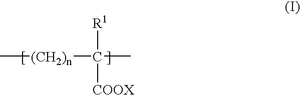
(in formula (I), n represents either 0 or 1, X represents a hydrogen atom, —NH₄, or 1/mM (wherein, M is a metal atom selected from the group consisting of alkali metals, alkali earth metals, transition metals, Zn and Cd, and m represents a valency of the metal), and RI represents a hydrogen atom or a methyl group) 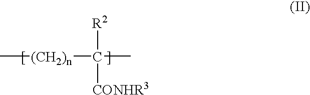
(in formula (II), n represents either 0 or 1, R² represents a hydrogen atom or a methyl group, and R³ represents a hydrogen atom, an alkyl group, alkenyl group, aralkyl group or phosphonoxyalkyl group of 1 to 18 carbon atoms).

13. A manufacturing method according to claim 12, wherein water is also mixed when the inorganic powder and the organic polymer are mixed to obtain the dental cement composition.
